(19) United States
(12) Patent Application Publication
(10) Pub. No.: US 2024/0342176 A1
(43) Pub. Date: Oct. 17, 2024

(54) PHARMACEUTICAL COMBINATION AND APPLICATION THEREOF

(71) Applicants: GUANGZHOU JOYO PHARMATECH CO., LTD, Guangzhou, Guangdong (CN); SHANGHAI JIA TAN PHARMATECH CO., LTD., Shanghai (CN)

(72) Inventors: Yongguo Li, Shanghai (CN); Wei Wei, Shanghai (CN); Wei Ye, Shanghai (CN)

(21) Appl. No.: 18/292,362

(22) PCT Filed: Jul. 27, 2022
(86) PCT No.: PCT/CN2022/108349

§ 371 Date: Jan. 26, 2024

(30) Foreign Application Priority Data
Jul. 27, 2021 (CN) 202110853024.1
Jul. 13, 2022 (CN) 202210828298.X

(51) Int. Cl. A61K 31/519; A61K 31/4439; A61K 39/00; A61P 35/00

(52) U.S. Cl. CPC A61K 31/519; A61K 31/4439; A61P 35/00; A61K 2039/505

(57) ABSTRACT

A pharmaceutical combination and an application thereof. The pharmaceutical combination comprises a PI3K inhibitor and an immune checkpoint inhibitor, wherein the PI3K inhibitor is selected from a compound represented by formula (I), linperlisib, samotolisib, copanlisib, SHC014748M, pilaralisib, buparlisib, taselisib, YZJ-0673, gedatolisib, omipalisib, bimiralisib, voxtalisib, AL58805, and HEC68498, and pharmaceutically acceptable salts thereof, and the immune checkpoint inhibitor is a PD-1/PD-L1 inhibitor. The compound represented by formula (Ia) has a high inhibitory effect on PI3Kδ and PI3Kγ kinases, and the pharmaceutical combination uses a PI3K inhibitor and a PD-1 inhibitor in combination, thereby effectively improving the inhibitory effect on tumors, and solving the problem of drug resistance of PD-1/PD-L1 inhibitors.

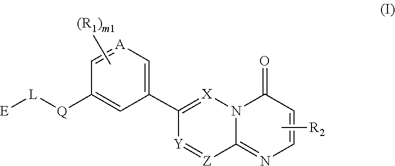

The present application claims the right of the priorities of Chinese patent application No. 2021108530241 filed on Jul. 27, 2021 and Chinese patent application No. 202210828298X filed on Jul. 13, 2022. The contents of the above Chinese patent applications are incorporated herein by reference in their entireties.

### TECHNICAL FIELD

The present disclosure belongs to the technical field of biomedicine and specifically relates to a pharmaceutical combination and a use thereof.

### BACKGROUND

Malignant tumors are one of the diseases with the largest mortality rate at present. Conventional treatment methods such as surgical resection, radiotherapy, and chemotherapy are widely used in the treatment of tumors, but at present, these methods have their limitations in the treatment of tumors and it is difficult to cure the tumors completely, especially for some metastatic malignant tumors. Programmed death 1 (PD-1) or programmed death-ligand 1 (PD-L1) and other immune checkpoint inhibitors are different from traditional treatment methods that directly eliminate tumors, but instead play a tumor-killing role by enhancing the body's own immune system function. At present, a variety of PD-1 targeted blocking antibodies (including Pembrolizumab, Nivolumab, etc.) approved by FDA have shown excellent therapeutic effects in a variety of solid tumors and hematological malignancies, and their greatest advantage is to produce a durable response in patients and bring long-term survival.

The mechanism of action of immune checkpoint inhibitors is as follows: PD-L1 on tumor cells interacts with PD-1 on T cells, reducing T cell function signals, thereby preventing the immune system from discovering and attacking tumor cells. Blocking the signal pathway between PD-L1 and PD-1 can prevent tumor cells from escaping the immune system in this way (as shown in FIG. 1, the picture is from Terese winslow in 2015), thereby achieving the effect of killing tumors.

At present, inhibitors targeting PD-1 and PD-L1, such as Nivolumab, Atezolizumab, Pembrolizumab, Durvalumab, etc., have achieved good results in the immunotherapy of malignant tumors such as melanoma, kidney cancer, and lung cancer.

Although inhibitors targeting the PD-1 target have achieved good results in the treatment of various malignant tumors, the shortcomings of this immunotherapy cannot be ignored. Firstly, the effective patient population ratio of PD-1 targeted inhibitors is low. In clinical practice, PD-1 inhibitors are only effective for about 20% of cancer patients. Secondly, for effective patients, drug resistance occurs after a period of medication. The main mechanisms of drug resistance include: immunosuppression of the tumor microenvironment, activation of other signaling pathways mediated by PD-L1 (such as STAT3), and activation of other immune checkpoints, etc. Tumor immunotherapy still faces many important obstacles at present. How to improve the effective rate of PD-1 targeted inhibitors and solve PD-1 drug resistance has become a hot spot in current immunotherapy research (see “Immuno-oncology agent IPI-549 is a modulator of P-glycoprotein (P-gp, MDR1, ABCB1)-mediated multidrug resistance (MDR) in cancer: In vitro and in vivo”).

Phosphatidylinositol 3 kinase (PI3K) plays an important role in cell growth, development, division, differentiation, and apoptosis, and is closely related to the occurrence and development of tumors. There are many subtypes of PI3K, wherein PI3Kα and PI3Kβ are expressed in various cells, while PI3Kδ and PI3Kγ are only expressed in the immune system. The signaling pathway composed of PI3K and its downstream molecule signaling protein kinase B (Akt)/mammalian target of rapamycin (mTOR) plays a key role in cell proliferation, survival, angiogenesis, and immune regulation. The FDA-approved PI3Kδ inhibitor idelalisib inhibits PI3Kδ with an IC₅₀ of 2.5 nM (reference: Lannutti B J, et al. CAL-101, a p110 delta selective phosphatidylinositol-3-kinase inhibitor for the treatment of B-cell malignancies, inhibits PI3K signaling and cellular viability, Blood, 2011, 117 (2), 591-594). Therefore, the inhibition of the signaling pathway mediated by PI3K will help to enhance the anti-tumor effect of the immune system and has broad application prospects.

### CONTENT OF THE PRESENT INVENTION

The technical problem to be solved by the present disclosure is to overcome the shortcomings of drug resistance and target inhibition efficiency of PD-1 inhibitors in the existing technology, and to provide a pharmaceutical combination and a use thereof. The present disclosure uses PI3K inhibitors and PD-1 inhibitors in combination, effectively improves the tumor inhibition effect of PD-1, and has good clinical application prospects.

The present disclosure solves the above problems through the following technical solutions.

The first aspect of the present disclosure provides a pharmaceutical combination, comprising a PI3K inhibitor and an immune checkpoint inhibitor;

the PI3K inhibitor is selected from a compound of formula (I), linperlisib, samotolisib, copanilisb, SHC014748M, pilaralisib, buparlisib, taselisib, YZJ-0673, gedatolisib, omipalisib, bimiralisib, voxtalisib, AL58805, HEC68498, and a pharmaceutically acceptable salt thereof; the immune checkpoint inhibitor is a PD-1/PD-L1 inhibitor;

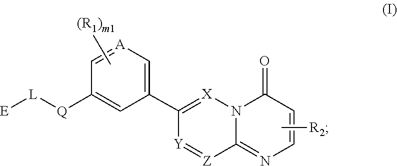

wherein E is C₃₋₁₀ heterocyclohydrocarbyl, C₃₋₁₀ cyclohydrocarbyl or C₁₋₆ alkyl optionally substituted by R₃;
L is —C(R₃)(R₃)—, —C(═O)N(Rₐ)—, —N(Rₐ)—, —C(═NRₐ)—, —S(═O)₂N(Rₐ)—, —S(═O)N(Rₐ)—, —O—, —S—, —C(═O)O—, —C(═O)—, —C(═S)—, —S(═O)—, —S(═O)₂—, or —N(Rₐ)C(═O)N(Rₐ)—, and Q is a single bond or —C(R₃)(R₃)—;
A is Nor C(R₃);
0 or 1 of X, Y, and Z is N, and the rest are C(R₃);
the “hetero” in the C₃₋₁₀ heterocyclohydrocarbyl represents a heteroatom or a heteroatom group, each independently being —C(═O)N(Rₐ)—, —N(Rₐ)—, —C(═NRₐ)—, —S(═O)₂N(Rₐ)—, —S(═O)N(Rₐ)—, —O—, —S—, —C(═O)O—, —C(═O)—, —C(═S)—, —S(═O)—, —S(═O)₂—, or —N(Rₐ)C(═O)N(Rₐ)—;

m₁ is 0, 1, 2, or 3;

R₁ to R₃ are each H, F, Cl, Br, I, CN, ORₐ, N(R_{b})(R_{c}), C₁₋₃ alkyl optionally substituted by R_{d},

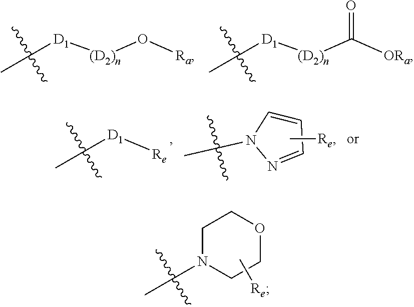

D₁ is a single bond, —C(Rₑ)(Rₑ)—, —C(═O)N(Rₐ)—, —N(Rₐ)—, —C(—NRₐ)—, —S(═O)₂N(Rₐ)—, —S(═O)N(Rₐ)—, —O—, —S—, —C(═O)O—, —C(═O)—, —C(═S)—, —S(═O)—, —S(═O)₂—, or —N(Rₐ)C(═O)N(Rₐ)—;
D₂ is —C(Rₐ)(Rₐ)—;
n is 1, 2, 3, 4, 5, or 6;
Rₐ, R_{b}, and R_{c} are each independently H, C₃₋₆ cycloalkyl or C₁₋₆ alkyl optionally substituted by Rₐ;
Rₑ is H, C₁₋₆ alkoxy or C₁₋₆ alkyl optionally substituted by Rₐ, or C₃₋₆ cycloalkoxy or C₃₋₆ cycloalkyl optionally substituted by Rₐ;
R_{d} is F, Cl, Br, I, CN, OH, CHO, COOH, CH₃, CF₃, CH₃O, or CH₃CH₂O, and the number of R_{d} is 0, 1, 2, or 3;
optionally, any two R₁, Rₐ and Rₐ in the same D₂, two D₂, or Rₐ and one D₂ are connected together to the same carbon atom or oxygen atom to form one or two 3-, 4-, 5-, or 6-membered carbon rings or oxygen heterocycles, wherein the number of oxygen atoms is 1 or 2.

In some embodiments of the present disclosure, the PI3K inhibitor is the compound of formula (I) or a pharmaceutically acceptable salt thereof, E is C₃₋₆ cycloalkyl or C₁₋₆ alkyl substituted by R₃, the number of R₃ is 0, 1, 2, or 3, or E is

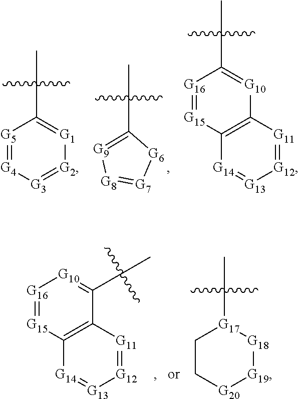

wherein,
0, 1, 2, or 3 of G₁-G₅ are N, and the rest are C(R₃);
G₆ is —C(R₃)(R₃)—, —C(═O)N(R₃)—, —N(R₃)—, —C(═NR₃)—, —S(═O)₂N(R₃)—, —S(═O)N(R₃)—, —O—, —S—, —C(═O)O—, —C(═O)—, —C(═S)—, —S(═O)—, —S(═O)₂—, or —N(R₃)C(═O)N(R₃)—;
0, 1, or 2 of G₇-G₉ are N, and the rest are C(R₃);
0, 1, 2, 3, or 4 of G₁₀-G₁₆ are N, and the rest are C(R₃); G₁₇ is N or C(R₃);
0, 1, 2, or 3 of G₁₈-G₂₂ are —C(═O)N(R₃)—, —N(R₃)—, —C(═NR₃)—, —S(═O)₂N(R₃)—, —S(═O)N(R₃)—, —O—, —S—, —C(═O)O—, —C(═O)—, —C(═S)—, —S(═O)—, —S(═O)₂—, or —N(R₃)C(═O)N(R₃)—, and the rest are —C(R₃)(R₃)—;
the other variables are defined as above.

In some specific embodiments of the present disclosure, the PI3K inhibitor is as shown in formula (Ia):

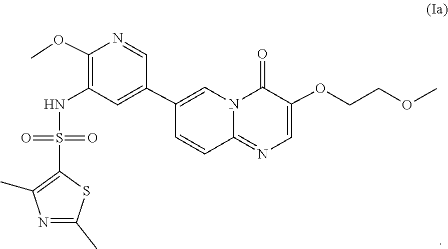

In the present disclosure, the PI3K inhibitor can also be conventional in this field, for example, an inhibitor targeting class I PI3K; the inhibitor targeting class I PI3K can be a pan-PI3K inhibitor or an inhibitor targeting specific subclasses of PI3Kα, PI3Kβ, PI3Kδ, or PI3Kγ.

In some embodiments of the present disclosure, the PD-1/PD-L1 inhibitor is a PD-1/PD-L1 antibody or an antigen-binding fragment thereof.

In some embodiments of the present disclosure, the PD-1/PD-L1 antibody is a murine antibody, a chimeric antibody, a humanized antibody, or a human antibody.

In some embodiments of the present disclosure, the PD-1 inhibitor is selected from Nivolumab, Pembrolizumab, Cemiplimab, Sintilimab, Camrelizumab, Tislelizumab, Atezolizumab, Avelumab, Durvalumab, Nofazinlimab (CS1003), MAX-10181, IMMH-010, INCB086550, RMP1-14, and GS-4224, and the PD-L1 inhibitor is selected from Atezolizumab, Durvalumab, Sugemalimab (CS1001), and Avelumab.

In some specific embodiments of the present disclosure, for the pharmaceutical combination, the PI3K inhibitor is selected from the compound of formula (I) and samotolisib; the PD-1 inhibitor is selected from Nivolumab, Pembrolizumab, Cemiplimab, Sintilimab, Camerelizumab, Tislelizumab, Atezolizumab, Avelumab, Durvalumab, CS1003, MAX-10181, IMMH-010, INCB086550, RMP1-14, and GS-4224; the PD-L1 inhibitor is selected from Atezolizumab, Durvalumab, Sugemalimab (CS1001), and Avelumab.

In some specific embodiments of the present disclosure, for the pharmaceutical combination, the PI3K inhibitor is the compound of formula (I), and the PD-1 inhibitor is Nivolumab.

In some specific embodiments of the present disclosure, for the pharmaceutical combination, the PI3K inhibitor is the compound of formula (Ia), and the PD-1 inhibitor is Nivolumab.

In the present disclosure, the antibody can be a complete antibody and any antigen-binding fragments thereof or a single chain thereof that specifically recognizes and binds to an antigen. Therefore, the term “antibody” includes proteins or peptides that contain at least a part of an immunoglobulin molecule with biological activity of binding to an antigen in a molecule. The “antigen-binding fragment” is a part of an antibody, such as F(ab′)₂, F(ab)₂, Fab′, Fab, Fv, and scFv.

In some embodiments of the present disclosure, the pharmaceutical combination further comprises a pharmaceutically acceptable carrier.

In the present disclosure, the pharmaceutically acceptable carrier can be conventional in this field, usually any type of non-toxic solid, semi-solid, or liquid filler, diluent, encapsulating material, or formulation adjuvant.

In some embodiments of the present disclosure, the pharmaceutically acceptable carrier is a pharmaceutical excipient.

The second aspect of the present disclosure provides a use of the pharmaceutical combination as defined in the first aspect in the manufacture of a medicament for treating a disease.

In some preferred embodiments of the present disclosure, the disease comprises a hematological malignant tumor or a solid malignant tumor.

In some more preferred embodiments of the present disclosure, the hematological malignant tumor is lymphoma; the solid malignant tumor is liver cancer or intestinal cancer.

In some specific embodiments of the present disclosure, the intestinal cancer is colon cancer or rectal cancer.

In the tumor microenvironment, cells such as regulatory T cells (Treg) and myeloid-derived suppressor cells (MDSC) create an immunosuppressive environment, significantly weakening the anti-tumor effect of the immune system. PI3Kδ inhibitors have a significant inhibitory effect on the proliferation of regulatory T cells (Treg cells) in the tumor microenvironment. And PI3Kγ is of great significance in the regulation of myeloid-derived suppressor cells (MDSC) in the tumor microenvironment. Therefore, PI3K inhibitors can solve the problem of drug resistance of PD-1/PD-L1 inhibitors and improve the effective rate of PD-1/PD-L1 targeted inhibitors by inhibiting the proliferation of immunosuppressive cells in the tumor microenvironment and regulating myeloid-derived suppressor cells in the tumor microenvironment.

The third aspect of the present disclosure provides a use of a pharmaceutical combination in the manufacture of a medicament for treating a disease; the pharmaceutical combination comprises a PI3K inhibitor and a PD-1/PD-L1 inhibitor; wherein the PD-1/PD-L1 inhibitor is as defined in the first aspect, the PI3K inhibitor is selected from eganelisib, idelalisib, and parsaclisib; the disease is as defined in the second aspect.

In some preferred embodiments of the present disclosure, the PD-1 inhibitor is selected from Nivolumab, Pembrolizumab, Cemiplimab, Sintilimab, Camrelizumab, Tislelizumab, Atezolizumab, Avelumab, Durvalumab, Nofazinlimab (CS1003), MAX-10181, IMMH-010, INCB086550, RMP1-14, and GS-4224, and the PD-L1 inhibitor is selected from Atezolizumab, Durvalumab, Sugemalimab (CS1001), and Avelumab.

The fourth aspect of the present disclosure provides a medicine box kit, comprising a medicine box A and a medicine box B; wherein the medicine box A comprises a PI3K inhibitor, and the medicine box B comprises an immune checkpoint inhibitor; the PI3K inhibitor and the immune checkpoint inhibitor are as defined in the first aspect or the third aspect.

In some embodiments of the present disclosure, the medicine box A and the medicine box B are used simultaneously or separately.

In some embodiments of the present disclosure, the medicine box kit further comprises a medicine box C, and the medicine box C comprises another therapeutic agent.

In some preferred embodiments of the present disclosure, the medicine box A, the medicine box B, and the medicine box C are used simultaneously or separately.

The therapeutic agent can be a therapeutic agent that has a synergistic effect with the PI3K inhibitor in the medicine box A and the immune checkpoint inhibitor in the medicine box B; for example, the therapeutic agent can be a cytokine/membrane protein antibody.

The fifth aspect of the present disclosure provides a kit, comprising the pharmaceutical combination as defined in the first aspect or the pharmaceutical combination in the use as defined in the third aspect.

The sixth aspect of the present disclosure provides a drug delivery device, comprising: (1) an infusion module for administering the pharmaceutical combination as defined in the first aspect or the pharmaceutical combination in the use as defined in the third aspect to a subject in need thereof, and (2) an optional pharmacodynamic monitoring module.

The seventh aspect of the present disclosure provides a method for treating a disease, comprising: administering the pharmaceutical combination as defined in the first aspect, the pharmaceutical combination in the use as defined in the third aspect, or the drug delivery device as defined in the sixth aspect to a subject in need thereof.

The disease is preferably as defined in the second aspect.

The eighth aspect of the present disclosure provides a pharmaceutical combination for use in treating a disease, and the pharmaceutical combination is the pharmaceutical combination as defined in the first aspect or the pharmaceutical combination in the use as defined in the third aspect.

The disease is preferably as defined in the second aspect.

On the basis of conforming to the common sense in this field, the above preferred conditions can be arbitrarily combined to obtain the preferred examples of the present disclosure.

The reagents and raw materials used in the present disclosure are all commercially available.

The positive progressive effects of the present disclosure are that:

The compound of formula (I) of the present disclosure has a high inhibitory effect on both PI3Kδ and PI3Kγ kinases; wherein the inhibitory effect of the compound of formula (I) on PI3Kδ is 13 or more times that of Idelalisib (which inhibits PI3Kδ with an IC₅₀ of 2.5 nM).

The pharmaceutical combination of the present disclosure, by combining the PI3K inhibitor and the PD-1 targeted inhibitor, effectively enhances the inhibitory effect on tumors and solves the problem of drug resistance to PD-1/PD-L1 inhibitors.

### BRIEF DESCRIPTION OF THE DRAWINGS

FIG. 1 is a schematic diagram of the background.

FIG. 2 is a schematic diagram of the results of Example 1.

FIG. 3 is a schematic diagram of the results of T-reg cells in the tumor of Example 2.

FIG. 4 is a schematic diagram of the results of M-MDSC cells in the tumor of Example 2.

FIG. 5 is a schematic diagram of the results of Example 4;

in the figure: A is compound I, and B is Idelalisib.

FIG. 6 is a schematic diagram of the results of Example 5.

FIG. 7 is a schematic diagram of the results of Example 6.

FIG. 8 is a schematic diagram of the results of Example 7.

FIG. 9 is a schematic diagram of the results of Example 8.

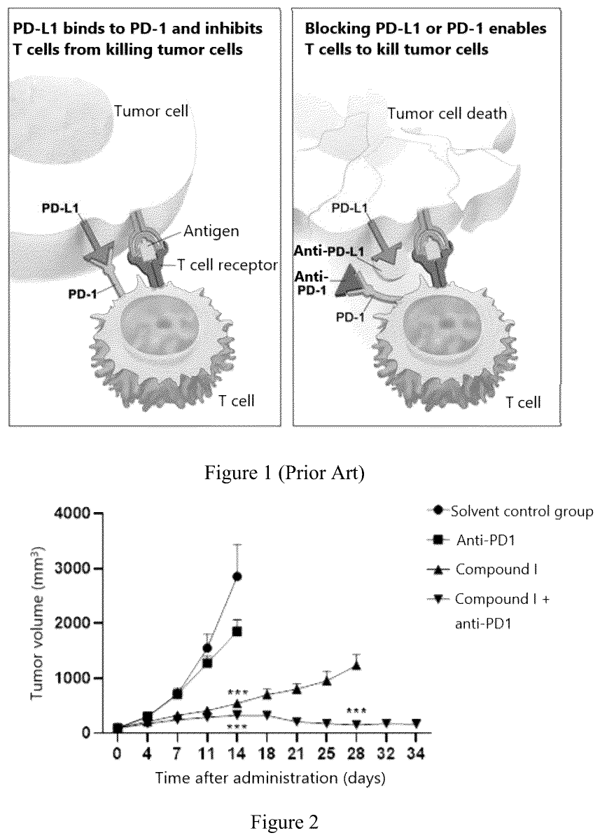

### DETAILED DESCRIPTION OF THE EMBODIMENT

The present disclosure will be further illustrated by way of examples below, but the present disclosure should not be limited to the scope of these examples. The experimental methods for which the specific conditions are not specified in the following examples are selected according to the conventional methods and conditions, or according to the commodity instructions.

### Example 1

1. Research purpose: To evaluate the anti-tumor effect of compound I in combination with anti-PD1 antibody in a subcutaneous homograft model of murine colon cancer CT26 cell line in female BalB/c mice.

Compound I is as shown in formula (Ia):

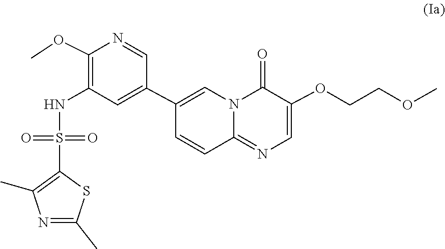

The preparation of compound I is referred to Chinese patent CN105461712B;
the anti-PD1 antibody used in this example is Leinco's anti-PD-1 (RMP1-14).
2. Experimental model: Subcutaneous homograft model of murine colon cancer CT26 cell line (purchased from ATCC CRL-2638) in female BalB/c mice.
3. Experimental animals: BalB/c mice, female, 6 to 7 weeks old (age of mice at the time of tumor cell inoculation), weighing 17.1 to 21.0 g, purchased from Jiangsu Jicui Yaokang Biotechnology Co., Ltd.
4. Cell culture: Murine colon cancer CT26 cells were cultured in vitro in monolayer, in RPMI1640 medium supplemented with 10% (v/v) fetal bovine serum, 100 U/mL penicillin, and 100 μg/mL streptomycin, at 37° C., 5% CO₂, 95% relative humidity, digested with trypsin twice a week for passage, and when the cells were in the logarithmic growth phase, the digested cells were used for inoculation.
5. Tumor inoculation: CT26 cells in the exponential growth phase were collected, resuspended in 0.2 mL of PBS to a suitable concentration, and used for subcutaneous tumor inoculation in mice. When the average tumor volume was about 100 mm³, the mice were randomly grouped according to tumor size.
6. Experimental methods:
CT26 cells were subcutaneously inoculated into BalB/c mice to establish a homograft tumor model. The experiment was divided into solvent control group, anti-PD1 antibody group, test drug compound I group, and combination group of test drug compound I plus anti-PD1 antibody, with 8 mice in each group. In the solvent control group, the drug was administered by intraperitoneal injection twice a week for a total of five times; the anti-PD1 antibody was administered by intraperitoneal injection twice a week for a total of five times; the test drug compound I was administered by oral gavage once a day; in the combination group of test drug compound I plus anti-PD1 antibody, the test drug compound I was administered by oral gavage once a day for a total of 35 days, at the same time, the anti-PD1 antibody was administered by intraperitoneal injection twice a week for a total of 10 times. After 32 days of administration, the tumor growth curve was obtained and analyzed (as shown in FIG. 2). The detailed dosage regimen is shown in Table 1.

TABLE 1
Dosage regimen

| | Number | | | Administration | | |
|---|---|---|---|---|---|---|
| | of | | Dose | volume | Route of | Frequency of |
| Group | animals | Drug | (mg/kg) | (μL/g) | administration | administration |
| 1 | 8 | Solvent | — | 10 | Intraperitoneal | Twice a week |
| | | control | | | | for a total of 5 |
| | | group | | | | times |
| 2 | 8 | anti-PD1^{a} | 10 | 10 | Intraperitoneal | Twice a week |
| | | | | | | for a total of 5 |
| | | | | | | times |
| 3 | 8 | Compound I^{b} | 0.2 | 10 | Gavage | Once a day for |
| | | | | | | 29 days |
| 4 | 8 | Compound I^{b} | 0.2 | 10 | Gavage | Once a day for |
| | | | | | | 35 days |
| | | anti-PD1^{a} | 10 | 10 | Intraperitoneal | Twice a week |
| | | | | | | for a total of |
| | | | | | | 10 times |

Note:
1) The solvent control group: normal saline;
2) ^{a}the solvent used is PBS;
3) ^{b}the solvent used is 1% DMSO + 99% (1% methylcellulose).

7. Experimental results: On the 14th day after grouping, there was no statistical difference (p=0.767) between the anti-PD1 group and the solvent control group, indicating this murine colon cancer CT26 tumor model showed drug resistance to anti-PD1 antibody. Compound I (p<0.001) alone and in combination with anti-PD1 (p<0.001) showed significant tumor inhibition difference compared with the control group in the murine colon cancer CT26 tumor model. On the 28th day after grouping, there was a significant tumor inhibition difference (p<0.001) between the combination group of compound I plus anti-PD1 and the compound I group.
8. Experimental conclusion: In the murine colon cancer CT26 tumor model resistant to anti-PD-1 antibody, by combining with compound I the efficacy of anti-PD1 antibody can be significantly enhanced.

### Example 2

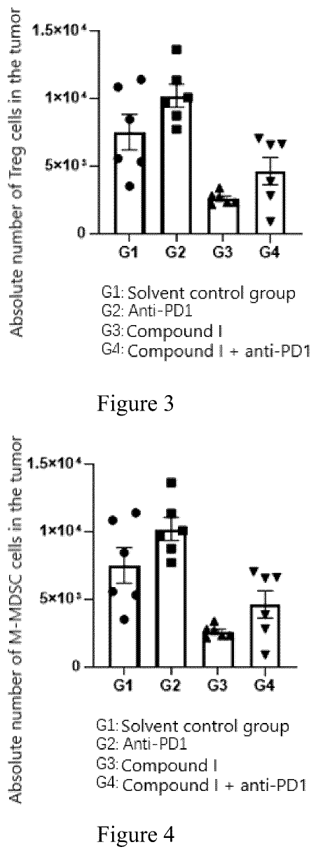

1. Research purpose: To explore the pharmacology of compound I in combination with anti-PD1 antibody in a tumor-bearing mouse model. The anti-PD1 antibody used in this example is Leinco's anti-PD-1 (RMP1-14).
2. Cell model: Subcutaneous homograft model of murine lymphoma A20 cell line in female BalB/c mice.
3. Experimental animals: BalB/c mice, female, 7 to 8 weeks old (age of mice at the time of tumor cell inoculation), with an average weight of 19.3 g, purchased from Shanghai Lingchang Biotechnology Co., Ltd.
4. Cell culture: Murine lymphoma A20 cells (purchased from ATCC TIB-208) were cultured in vitro in monolayer, in RPMI1640 medium supplemented with 10% fetal bovine serum, 100 U/mL penicillin, and 100 μg/mL streptomycin, at 37° C., 5% CO₂, 95% relative humidity, digested with trypsin twice a week for passage, and when the cells were in the logarithmic growth phase, the digested cells were used for inoculation.
5. Tumor inoculation: A20 cells in the exponential growth phase were collected, resuspended in 0.2 mL of PBS to a suitable concentration, and used for subcutaneous tumor inoculation in mice. When the average tumor volume was about 100 mm³, the mice were randomly grouped according to tumor size.
6. Experimental methods:
1) A20 cells were subcutaneously inoculated into BalB/c mice to establish a homograft tumor model. The experiment was divided into solvent control group, anti-PD1 antibody group, test drug compound I group, and combination group of compound I plus anti-PD1 antibody. The anti-PD1 antibody was administered by intraperitoneal injection twice a week, and the test drug compound I was administered by oral gavage once a day. The solvent and specific dosage regimen are shown in Table 4 and the note section. Seven days after administration in groups, tumors from each group were taken for flow cytometry (FACS) to analyze the absolute cell number of immune cells, including MDSC, Treg, etc.
2) Antibody information: CD45 (purchased from Biolegend), CD3 (purchased from BD), CD4 (purchased from Biolegend), CD8 (purchased from eBiosciences), Foxp3 (purchased from eBiosciences), CD11b (purchased from Biolegend), F4/80 (purchased from Biolegend), I-A/I-E (purchased from Biolegend), CD206 (purchased from Biolegend), Ly-6G (purchased from BD), Ly-6C (purchased from Biolegend), CD19 (purchased from Biolegend), CD25 (purchased from BD), and L/D (purchased from eBiosciences).
7. Experimental results:
On the seventh day after grouping, compared with the anti-PD1 treatment group, the combination treatment of compound I and anti-PD1 antibody significantly reduced the Treg cells in the mouse tumor (P=0.0022). Compared with the solvent control group and anti-PD1 monotherapy group, the compound I treatment group could significantly reduce the M-MDSC cells in the tumor (p=0.0022 and P=0.0087). The specific experimental results are shown in FIGS. 3 and 4.
5. Experimental conclusion:
This murine lymphoma A20 tumor model shows drug resistance to anti-PD1 antibody. In the subcutaneous homograft BalB/c mouse model, compound I can significantly inhibit the immunosuppressive cells Treg and M-MDSC in the tumor.

### Example 3

1. Research purpose: The inhibitory effect of compound I on the enzymatic activity of PI3Kδ and PI3Kγ in vitro.
2. Experimental materials:
(1) Main instrument: Envision (PerkinElmer-2104)
(2) Main reagents: ADP-Glo kinase kit (purchased from Promega), PI3Kδ (P1108/P85a) (purchased from Millipore), and PI3Kγ (P120γ) (purchased from Millipore).
3. Experimental methods:
1) Preparation of buffer salt solution: A 10× buffer salt solution with a pH of 7.5 containing 500 mM HEPES, 500 mM NaCl, and 30 mM MgCl₂ was prepared with ultrapure water and stored at 4° C. for later use. Before use, the above buffer salt solution was diluted to a 3.33× buffer salt solution and BSA was added to a final concentration of 0.333 mg/mL.
2) A 100× reference compound (compound I) was prepared with a starting concentration of 100 nM, diluted in a 3-fold serial dilution to 10 concentrations and transferred to the corresponding 384 microwell plate at 50 nL/well. In the control group, 50 nL/well of DMSO was added.
3) 3.33×PI3K solutions were prepared with the 3.33× buffer salt solution, PI3Kδ at a final concentration of 0.25 nM, PI3Kγ at a final concentration of 0.4 nM. A 3.33×PIP2:3PS solution was prepared, mixed with the enzyme solutions at a volume ratio of 1:1, and added to the 384 microwell plate in 3 μL/well. The mixed solution of buffer salt solution/PIP2:3PS was added in the complete inhibition control group. The mixture was mixed well, centrifuged, and incubated for 20 minutes at 23° C.
4) The 384 microwell plate was removed, and 2.5×ATP solutions, at a final concentration of 40 μM (PI3Kδ) and 25 μM (PI3Kγ) respectively, were prepared with ultrapure water, added to the 384 microwell plate in 2 μL/well, mixed well, centrifuged, and incubated for 120 minutes at 23° C. Then, 5 L/well of ADP-Glo reagent was added, mixed well, centrifuged, and incubated for 60 minutes at 23° C. 10 μL/well of kinase detection reagent was added, mixed well, centrifuged, and incubated for 30 minutes at 23° C. The luminescence value was read using Envision.
4. Experimental results:
The experiment used the ADP-Glo chemiluminescence method as the enzymatic activity detection method to determine the inhibitory effect of the test compound I on the enzymatic activity of PI3Kδ and PI3Kγ. The detection results are shown in Table 2.

TABLE 2
Detection results of the inhibitory activity of
compound I on PI3K kinases (IC₅₀, mean ± SD)

| | Name of compound |
|---|---|
| | Compound I |
| Enzyme | (N = 3) |
| PI3Kδ IC₅₀ (nM) | 0.18 ± 0.01 |
| PI3Kγ IC₅₀ (nM) | 0.29 ± 0.02 |

5. Experimental conclusion: This experiment determines the inhibitory effect of the test compound I on the enzymatic activity of two PI3K kinases, PI3Kδ and PI3Kγ. The experimental results show that compound I has a high inhibitory effect on both PI3Kδ and PI3Kγ kinases.

### Example 4

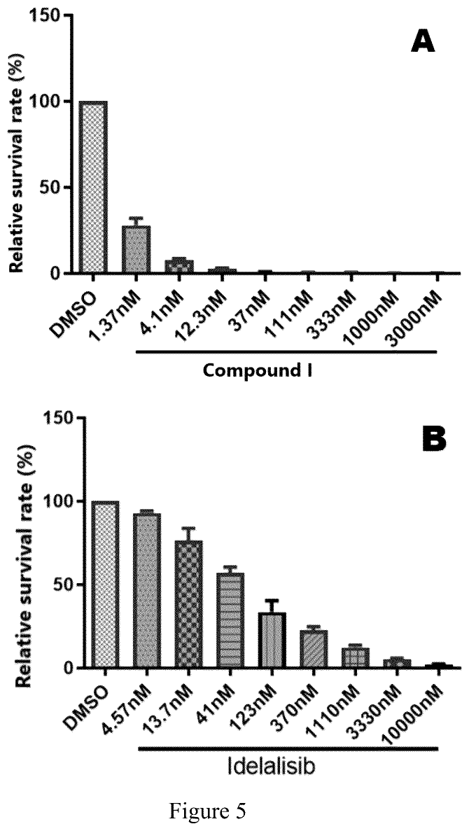

1. Research purpose: The in vitro pharmacodynamic experiment of compound I on human Treg cells.
2. Experimental materials: Human naïve CD4 isolation kit (purchased from Stem cell), X-VIVO medium (purchased from Lonza Bioscience), anti-human CD3 (purchased from eBioscience), anti-human CD28 (purchased from eBioscience), Human IL-2 protein (purchased from R&D Systems), TGF-b1 (purchased from R&D Systems), Live/Dead Fixable Near-IR Dead Cell Stain Kit (purchased from Life technologies), anti-Human CD4 (purchased from BD Biosciences), anti-Human CD25 (purchased from BD Horizon), anti-Human Foxp3 (purchased from BD Biosciences), Idelalisib (purchased from Shanghai Taosu Biochemical Technology Co., Ltd.).
3. Experimental methods:
1) A 96-well cell culture plate was coated with 10 μg/mL anti-human CD3, 50 μL per well, incubated at 37° C. for three hours, and then rinsed with X-VIVO medium.
2) Human peripheral blood mononuclear cells (purchased from Hemacare) were resuscitated and stained with CellTrace Violet (CTV).
3) After staining, Human Naive CD4+ T cells were isolated using the Human naïve CD4 isolation kit.
4) IL-2 (10 ng/ml), CD28 (2 μg/mL), and TGF-b (1 ng/mL) were added to the cell culture medium, and different concentrations of the test compounds were added at the same time.
5) After five days of compound incubation, CD4, CD25, and Foxp3 were detected by flow cytometry. The positive counts of CD4, CD25, and Foxp3 were calculated, and the relative survival rate and IC₅₀ were calculated compared with the DMSO control group.
4. Experimental results:
The experiment used the flow cytometry method to determine the in vitro pharmacodynamic experiment of the test compound I on human Treg cells. The detection results are shown in A and B of FIG. 5 and Table 3.

TABLE 3
Detection results of the inhibitory activity
of the compound on human Treg cells (IC₅₀)

| Compound | IC₅₀ (nM) |
|---|---|
| Compound I | 0.01 |
| Idelalisib | 31.72 |

5. Experimental conclusion: This experiment determines the inhibitory effect of the test compound I and Idelalisib on the activity of human Treg cells. The experimental results show that compound I has a significant inhibitory effect on the activity of human Treg cells with an IC₅₀ of 0.01 nM. Compared with the PI3Kδ inhibitor Idelalisib, the inhibitory activity of compound I on human Treg cells is 3172 times that of Idelalisib.

### Example 5

1. Research purpose: To evaluate the anti-tumor effect of compound I in combination with humanized anti-PD1 antibody drug Nivolumab in a subcutaneous homograft model of murine colon cancer CT26 cell line in humanized hPD-1 sKI HuGEMM BalB/c mice.

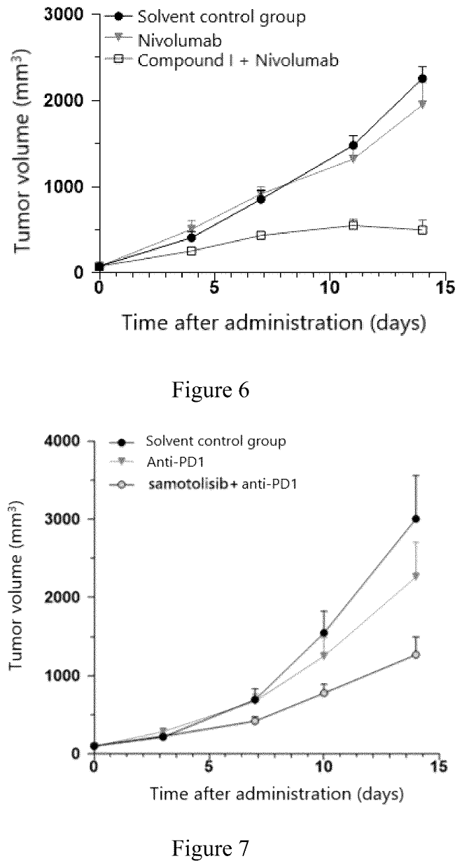

The source of the anti-PD1 antibody: The source of the humanized anti-PD1 antibody drug Nivolumab (purchased from Opdivo, batch: ACA4299).
2. Experimental model: Subcutaneous homograft model of murine colon cancer CT26 cell line (purchased from ATCC CRL-2638) in female hPD-1 sKI HuGEMM BalB/c mice.
3. Experimental animals: hPD-1 sKI HuGEMM BALB/c mice, female, 6 to 8 weeks old (age of mice at the time of tumor cell inoculation), weighing 17.1 to 21.0 g, purchased from Jiangsu Jicui Yaokang Biotechnology Co., Ltd.
4. Cell culture: Murine colon cancer CT26 cells were cultured in vitro in monolayer, in RPMI1640 medium supplemented with 10% (v/v) fetal bovine serum, 100 U/mL penicillin, and 100 μg/mL streptomycin, at 37° C., 5% CO₂, 95% relative humidity, digested with trypsin twice a week for passage, and when the cells were in the logarithmic growth phase, the digested cells were used for inoculation.
5. Tumor inoculation: CT26 cells in the exponential growth phase were collected, resuspended in 0.1 mL of PBS to a suitable concentration, and used for subcutaneous tumor inoculation in mice. When the average tumor volume was about 80 mm³, the mice were randomly grouped according to tumor size.
6. Experimental methods:
CT26 cells were subcutaneously inoculated into hPD-1 sKI HuGEMM BalB/c mice to establish a homograft tumor model. The experiment was divided into solvent control group, humanized anti-PD1 antibody (Nivolumab) group, and combination group of test drug compound I plus humanized anti-PD1 antibody (Nivolumab), with 5 mice in each group. The detailed dosage regimen is shown in Table 4. After 14 days of administration, the tumor growth curve was obtained and analyzed (as shown in FIG. 6).

TABLE 4
Dosage regimen

| | Number | | | Administration | | |
|---|---|---|---|---|---|---|
| | of | | Dose | volume | Route of | Frequency of |
| Group | animals | Drug | (mg/kg) | (μL/g) | administration | administration |
| 1 | 5 | Solvent | — | 10 | Intraperitoneal | Twice a week |
| | | control | | | | for a total |
| | | group | | | | of 5 times |
| 2 | 5 | Nivolumab^{a} | 10 | 10 | Intraperitoneal | Twice a week |
| | | | | | | for a total |
| | | | | | | of 5 times |
| 3 | 5 | Compound I^{b} | 0.2 | 10 | Gavage | Once a day |
| | | | | | | for 15 days |
| | | Nivolumab^{a} | 10 | 10 | Intraperitoneal | Twice a week |
| | | | | | | for a total |
| | | | | | | of 5 times |

Note:
1) The solvent control group: normal saline;
2) ^{a}the solvent used is PBS;
3) ^{b}the solvent used is 1% DMSO + 99% (1% methylcellulose).

7. Experimental results: On the 14th day after grouping, there was no statistical difference (p=0.478) between the Nivolumab group and the solvent control group, indicating this murine colon cancer CT26 tumor model showed drug resistance to Nivolumab antibody. Compound I in combination with Nivolumab (p<0.001) showed a significant tumor inhibition difference compared with the control group in the murine colon cancer CT26 tumor model. There was a significant tumor inhibition difference (p<0.001) between the combination group of drug compound I plus Nivolumab and the Nivolumab treatment group.
8. Experimental conclusion: In the murine colon cancer CT26 tumor model resistant to Nivolumab antibody in hPD-1 sKI Hu GEMM BALB/c mice, by combining with compound I the efficacy of Nivolumab antibody can be significantly enhanced.

### Example 6

1. Research purpose: To evaluate the anti-tumor effect of samotolisib (LY3023414, CAS number: 1386874-06-1) in combination with murine anti-PD1 antibody in a subcutaneous homograft model of murine lymphoma A20 cell line in BALB/c mice.

The anti-PD1 antibody used in this example is Leinco's anti-PD-1 (RMP1-14).
2. Experimental model: Subcutaneous homograft model of murine lymphoma A20 cell line (purchased from ATCC TIB-208) in female BalB/c mice.
3. Experimental animals: BALB/c mice, female, 6 to 7 weeks old (age of mice at the time of tumor cell inoculation), weighing 16.8 to 20.6 g, purchased from Shanghai Lingchang Biotechnology Co., Ltd.
4. Cell culture: Murine lymphoma A20 cells were cultured in vitro in monolayer, in RPMI1640 medium supplemented with 10% (v/v) fetal bovine serum, 100 U/mL penicillin, and 100 μg/mL streptomycin, at 37° C., 5% CO₂, 95% relative humidity, digested with trypsin twice a week for passage, and when the cells were in the logarithmic growth phase, the digested cells were used for inoculation.
5. Tumor inoculation: A20 cells in the exponential growth phase were collected, resuspended in 0.1 mL of PBS to a suitable concentration, and used for subcutaneous tumor inoculation in mice. When the average tumor volume was about 100 mm³, the mice were randomly grouped according to tumor size.
6. Experimental methods:
A20 cells were subcutaneously inoculated into BalB/c mice to establish a homograft tumor model. The experiment was divided into solvent control group, anti-PD1 antibody group, combination group of test drug samotolisib plus anti-PD1 antibody, with 5 mice in each group. The detailed dosage regimen is shown in Table 6. The tumor growth curve was obtained and analyzed (as shown in FIG. 7).

TABLE 6
Dosage regimen

| | Number | | | Administration | | |
|---|---|---|---|---|---|---|
| | of | | Dose | volume | Route of | Frequency of |
| Group | animals | Drug | (mg/kg) | (μL/g) | administration | administration |
| 1 | 5 | Solvent | — | 10 | Intraperitoneal | Twice a week for a |
| | | control | | | | total of 5 times |
| 2 | 5 | anti-PD1^{a} | 10 | 10 | Intraperitoneal | Twice a week for a |
| | | | | | | total of 5 times |
| 3 | 5 | Samotolisib^{b} | 0.2 | 10 | Gavage | Once a day for 15 |
| | | | | | | days |
| | | anti-PD1^{a} | 10 | 10 | Intraperitoneal | Twice a week for a |
| | | | | | | total of 5 times |

Note:
1) The solvent control group: normal saline;
2) ^{a}the solvent used is PBS;
3) ^{b}the solvent used is a 0.01N HCl solution of 2% (w/v) PVP K30.

7. Experimental results: On the 14th day after grouping, there was no statistical difference (p=0.841) between the murine anti-PD1 group and the solvent control group, indicating this murine lymphoma A20 tumor model showed drug resistance to murine anti-PD1 antibody. Samotolisib in combination with murine anti-PD1 showed a significant tumor inhibition difference (P<0.05) compared with the control group in the murine lymphoma A20 tumor model. The average tumor volume of the solvent control group was 3005.01 mm³, and the average tumor volumes of the anti-PD-1 treatment group and the combination group of samotolisib plus anti-PD-1 treatment were 2169.65 mm³ and 1268.94 mm³, respectively, and the relative tumor inhibition rates TGI (%) were 25.33% and 57.75%, respectively.
8. Experimental conclusion: In the murine lymphoma A20 tumor homograft model in BALB/c mice, by combining with samotolisib (LY3023414) the efficacy of anti-PD1 antibody can be significantly enhanced.

### Example 7

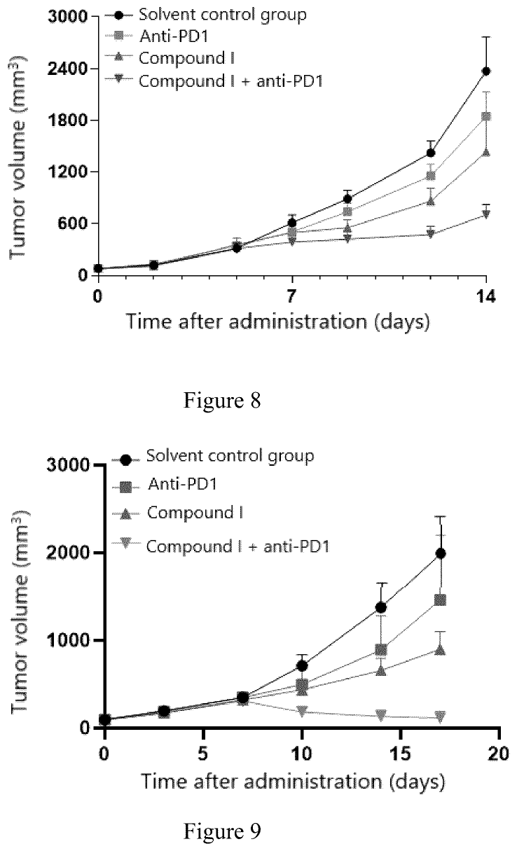

1. Research purpose: To evaluate the anti-tumor effect of compound I in combination with anti-PD1 antibody in a subcutaneous homograft model of murine liver cancer H22 cell line in female BalB/c mice.
2. Experimental model: Subcutaneous homograft model of murine liver cancer H22 cell line (CCTCC, GDC091) in female BalB/c mice. The anti-PD1 antibody used in this example is an anti-PD1 antibody purchased from BioXcell.
3. Experimental animals: BalB/c mice, female, 6 to 8 weeks old (age of mice at the time of tumor cell inoculation), weighing 17 to 20 g, purchased from Zhejiang Vital River Laboratory Animal Technology Co., Ltd.
4. Cell culture: Murine liver cancer H22 cells were cultured in vitro in monolayer, in RPMI1640 medium supplemented with 10% (v/v) fetal bovine serum, 100 U/mL penicillin, and 100 μg/mL streptomycin, at 37° C., 5% CO₂, 95% relative humidity, digested with trypsin twice a week for passage, and when the cells were in the logarithmic growth phase, the digested cells were used for inoculation.
5. Tumor inoculation: H22 cells in the exponential growth phase were collected, resuspended in 0.1 mL of PBS to a suitable concentration, and used for subcutaneous tumor inoculation in mice. When the average tumor volume was about 80 mm³, the mice were randomly grouped according to tumor size.
6. Experimental method: H22 cells were subcutaneously inoculated into BalB/c mice to establish a homograft tumor model. The experiment was divided into solvent control group, anti-PD1 antibody group, test drug compound I group, and combination group of test drug compound I plus anti-PD1 antibody, with 6 mice in each group. After 15 times of administration, the tumor growth curve was obtained and analyzed (as shown in FIG. 8). The detailed dosage regimen is shown in Table 7.

TABLE 7
Dosage regimen

| | Number | | | Administration | | |
|---|---|---|---|---|---|---|
| | of | | Dose | volume | Route of | Frequency of |
| Group | animals | Drug | (mg/kg) | (μL/g) | administration | administration |
| 1 | 6 | Solvent | — | 10 | Intraperitoneal | Twice a week |
| | | control | | | | for a total of 5 |
| | | group | | | | times |
| 2 | 6 | anti-PD1^{a} | 10 | 10 | Intraperitoneal | Twice a week |
| | | | | | | for a total of 5 |
| | | | | | | times |
| 3 | 6 | Compound I^{b} | 0.2 | 10 | Gavage | Once a day for |
| | | | | | | 15 times |
| 4 | 6 | Compound I^{b} | 0.2 | 10 | Gavage | Once a day for |
| | | | | | | 15 times |
| | | anti-PD1^{a} | 10 | 10 | Intraperitoneal | Twice a week |
| | | | | | | for a total of 5 |
| | | | | | | times |

Note:
1) The solvent control group: normal saline;
2) ^{a}the solvent used is PBS;
3) ^{b}the solvent used is 1% DMSO + 99% (1% methylcellulose).

7. Experimental results:
On the 14th day after grouping, compared with the solvent control group, the anti-PD1 group and the compound I group showed no statistical difference (p=0.712 and P=0.409), indicating this murine liver cancer (H22) tumor model showed drug resistance to anti-PD1 antibody. There was a significant tumor inhibition difference (P=0.027) between the combination group of compound I plus anti-PD1 and the control group.
8. Experimental conclusion: In the murine liver cancer H22 tumor model resistant to anti-PD-1 antibody, by combining with compound I the efficacy of anti-PD1 antibody can be significantly enhanced.

### Example 8

1. Research purpose: To evaluate the anti-tumor effect of compound I in combination with anti-PD1 antibody in a subcutaneous homograft model of murine lymphoma A20 cell line in female BalB/c mice.

The anti-PD1 antibody used in this example is Leinco's anti-PD-1 (RMP1-14).
2. Experimental model: Subcutaneous homograft model of murine lymphoma A20 cells (derived from ATCC, TIB-208) in female BalB/c mice.
3. Experimental animals: BalB/c mice, female, 6 to 7 weeks old (age of mice at the time of tumor cell inoculation), with an average weight of 17.6 g, purchased from Beijing Vital River Laboratory Animal Technology Co., Ltd.
4. Cell culture: Murine lymphoma A20 cells were cultured in vitro in monolayer, in RPMI1640 medium supplemented with 10% fetal bovine serum, 100 U/mL penicillin, and 100 μg/mL streptomycin, at 37° C., 5% CO₂, 95% relative humidity, digested with trypsin twice a week for passage, and when the cells were in the logarithmic growth phase, the digested cells were used for inoculation.
5. Tumor inoculation: A20 cells in the exponential growth phase were collected, resuspended in 0.2 mL of PBS to a suitable concentration, and used for subcutaneous tumor inoculation in mice. When the average tumor volume was about 100 mm³, the mice were randomly grouped according to tumor size.
6. Experimental methods:
A20 cells were subcutaneously inoculated into BalB/c mice to establish a homograft tumor model. The experiment was divided into solvent control group, anti-PD1 antibody group, test drug compound I group, and combination group of test drug compound I plus anti-PD1 antibody group, with 6 mice in each group. The tumor growth curve is shown in FIG. 9. The detailed dosage regimen is shown in Table 8.

TABLE 8
Dosage regimen

| | Number | | | Administration | | |
|---|---|---|---|---|---|---|
| | of | | Dose | volume | Route of | Frequency of |
| Group | animals | Drug | (mg/kg) | (μL/g) | administration | administration |
| 1 | 6 | Solvent | — | 10 | Intraperitoneal | Twice a week |
| | | control | | | | for a total of 6 |
| | | group | | | | times |
| 2 | 6 | anti-PD1^{a} | 10 | 10 | Intraperitoneal | Twice a week |
| | | | | | | for a total of 6 |
| | | | | | | times |
| 3 | 6 | Compound I^{b} | 0.2 | 10 | Gavage | Once a day for |
| | | | | | | 18 times |
| 4 | 6 | Compound I^{b} | 0.2 | 10 | Gavage | Once a day for |
| | | | | | | 18 times |
| | | anti-PD1^{a} | 10 | 10 | Intraperitoneal | Twice a week |
| | | | | | | for a total of 6 |
| | | | | | | times |

Note:
1) The solvent control group: normal saline;
2) ^{a}the solvent used is PBS;
3) ^{b}the solvent used is 1% DMSO + 99% (1% methylcellulose).

7. Experimental results:
On the 17th day after grouping, compared with the solvent control group, the anti-PD1 group and the compound I group showed no significant difference (p=0.461 and 0.352), indicating this murine lymphoma A20 tumor model showed drug resistance to anti-PD1 antibody and compound I. Compound I in combination anti-PD-1 showed a statistically significant difference (p=0.004) compared with the control group.
8. Experimental conclusion: In the murine lymphoma A20 tumor model resistant to anti-PD-1 antibody, by combining with compound I the efficacy of anti-PD1 antibody can be significantly enhanced.

Although specific embodiments of the present disclosure have been described above, those skilled in the art should understand that these are only examples, and various changes or modifications can be made to these embodiments without departing from the principles and essence of the present disclosure. Therefore, the scope of protection for the present disclosure is defined by the appended claims.

1. A pharmaceutical combination, comprising a PI3K inhibitor and an immune checkpoint inhibitor;
wherein the PI3K inhibitor is a compound of formula (I) or a pharmaceutically acceptable salt thereof; and
the immune checkpoint inhibitor is a PD-1/PD-L1 inhibitor; 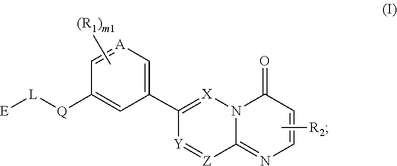
wherein E is C₃₋₁₀ heterocyclohydrocarbyl, C₃₋₁₀ cyclohydrocarbyl or C₁₋₆ alkyl optionally substituted by R₃;
L is —C(R₃)(R₃)—, —C(═O)N(Rₐ)—, —N(Rₐ)—, —C(═NRₐ)—, —S(═O)₂N(Rₐ)—, —S(═O)N(Rₐ)—, —O—, —S—, —C(═O)O—, —C(═O)—, —C(═S)—, —S(═O)—, —S(═O)₂—, or —N(Rₐ)C(═O)N(Rₐ)—;
Q is a single bond or —C(R₃)(R₃)—;
A is N or C(R₃);
0 or 1 of X, Y, and Z is N, and the rest are C(R₃);
the “hetero” in the C₃₋₁₀ heterocyclohydrocarbyl represents a heteroatom or a heteroatom group, each independently being —C(═O)N(Rₐ)—, —N(Rₐ)—, —C(═NRₐ)—, —S(═O)₂N(Rₐ)—, —S(═O)N(Rₐ)—, —O—, —S—, —C(═O)O—, —C(═O)—, —C(═S)—, —S(═O)—, —S(═O)₂—, or —N(Rₐ)C(═O)N(Rₐ)—;
m₁ is 0, 1, 2, or 3;
R₁ to R₃ are each H, F, Cl, Br, I, CN, ORₐ, N(R_{b})(R_{c}), C₁₋₃ alkyl optionally substituted by R_{d}, 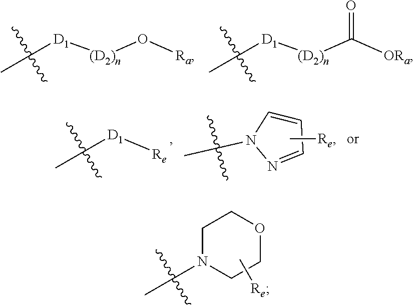
D₁ is a single bond, —C(Rₑ)(Rₑ)—, —C(═O)N(Rₐ)—, —N(Rₐ)—, —C(═NRₐ)—, —S(═O)₂N(Rₐ)—, —S(═O)N(Rₐ)—, —O—, —S—, —C(═O)O—, —C(═O)—, —C(═S)—, —S(═O)—, —S(═O)₂—, or —N(Rₐ)C(═O)N(Rₐ)—;
D₂ is —C(Rₐ)(Rₐ)—;
n is selected from 1, 2, 3, 4, 5, or 6;
Rₐ, R_{b}, and R_{c} are each independently H, C₃₋₆ cycloalkyl or C₁₋₆ alkyl optionally substituted by Rₐ;
Rₑ is H, C₁₋₆ alkoxy or C₁₋₆ alkyl optionally substituted by R_{d}, or C₃₋₆ cycloalkoxy or C₃₋₆ cycloalkyl optionally substituted by R_{d};
R_{d} is F, Cl, Br, I, CN, OH, CHO, COOH, CH₃, CF₃, CH₃O, or CH₃CH₂O, and the number of R_{d} is 0, 1, 2, or 3;
optionally, any two R₁, Rₐ and Rₐ in the same D₂, two D₂, or Rₐ and one D₂ are connected together to the same carbon atom or oxygen atom to form one or two 3-, 4-, 5-, or 6-membered carbon rings or oxygen heterocycles, wherein the number of oxygen atoms is 1 or 2.

2. The pharmaceutical combination according to claim 1, wherein
E is C₃₋₆ cycloalkyl or C₁₋₆ alkyl substituted by R₃, the number of R₃ is 0, 1, 2, or 3, or E is 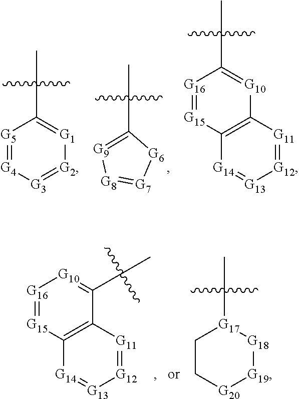
wherein
0, 1, 2, or 3 of G₁-G₅ are N, and the rest are C(R₃);
G₆ is —C(R₃)(R₃)—, —C(═O)N(R₃)—, —N(R₃)—, —C(═NR₃)—, —S(═O)₂N(R₃)—, —S(═O)N(R₃)—, —O—, —S—, —C(═O)O—, —C(═O)—, —C(═S)—, —S(═O)—, —S(═O)₂—, or —N(R₃)C(═O)N(R₃)—;
0, 1, or 2 of G₇-G₉ are N, and the rest are C(R₃);
0, 1, 2, 3, or 4 of G₁₀-G₁₆ are N, and the rest are C(R₃);
G₁₇ is N or C(R₃);
0, 1, 2, or 3 of G₁₈-G₂₂ are —C(═O)N(R₃)—, —N(R₃)—, —C(═NR₃)—, —S(═O)₂N(R₃)—, —S(═O)N(R₃)—, —O—, —S—, —C(═O)O—, —C(═O)—, —C(═S)—, —S(═O)—, —S(═O)₂—, or —N(R₃)C(═O)N(R₃)—, and the rest are —C(R₃)(R₃)—.

3-10. (canceled)

11. The pharmaceutical combination according to claim 1, wherein the PI3K inhibitor is a compound of formula (Ia) or a pharmaceutically acceptable salt thereof;

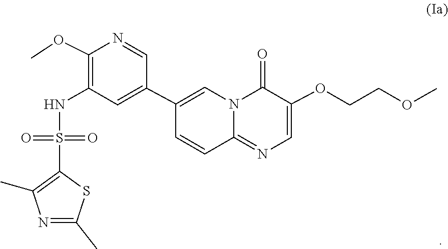

12. The pharmaceutical combination according to claim 1, wherein the PD-1/PD-L1 inhibitor is a PD-1/PD-L1 antibody or an antigen-binding fragment thereof.

13. The pharmaceutical combination according to claim 12, wherein the PD-1/PD-L1 antibody is a murine antibody, a chimeric antibody, a humanized antibody, or a human antibody.

14. The pharmaceutical combination according to claim 1, wherein the PD-1/PD-L1 inhibitor is selected from the group consisting of Nivolumab, Pembrolizumab, Cemiplimab, Sintilimab, Camerelizumab, Tislelizumab, Atezolizumab, Avelumab, Durvalumab, CS1003, MAX-10181, IMMH-010, INCB086550, RMP1-14, GS-4224, and CS1001.

15. The pharmaceutical combination according to claim 1, wherein the PD-1/PD-L1 inhibitor is a PD-1/PD-L1 antibody; and
the PI3K inhibitor is a compound of formula (Ia) or a pharmaceutically acceptable salt thereof;

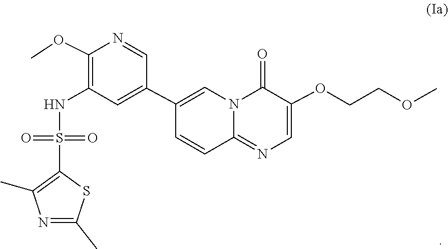

16. The pharmaceutical combination according to claim 15, wherein the PD-1/PD-L1 inhibitor is selected from the group consisting of Nivolumab, Pembrolizumab, Cemiplimab, Sintilimab, Camerelizumab, Tislelizumab, Atezolizumab, Avelumab, Durvalumab, CS1003, RMP1-14, and CS1001.

17. The pharmaceutical combination according to claim 15, wherein the PD-1/PD-L1 inhibitor is Nivolumab or RMP1-14.

18. The pharmaceutical combination according to claim 15, wherein the PD-1/PD-L1 inhibitor is Nivolumab.

19. The pharmaceutical combination according to claim 15, further comprising a pharmaceutically acceptable carrier.

20. A method of treating a hematological malignant tumor in a subject in need thereof, comprising administering a therapeutically effective amount of the pharmaceutical combination as defined in claim 1 to the subject.

21. The method according to claim 20, wherein the hematological malignant tumor is lymphoma.

22. A method of treating a solid malignant tumor in a subject in need thereof, comprising administering a therapeutically effective amount of the pharmaceutical combination as defined in claim 1 to the subject.

23. The method according to claim 22, wherein the hematological malignant tumor is liver cancer or intestinal cancer.

24. The method according to claim 23, wherein the intestinal cancer is colon cancer.

25. A medicine box kit, comprising a medicine box A and a medicine box B; wherein the medicine box A comprises a PI3K inhibitor, and the medicine box B comprises a PD-1/PD-L1 inhibitor; the PI3K inhibitor and the PD-1/PD-L1 inhibitor are as defined in claim 1.

26. The medicine box kit according to claim 25, wherein the medicine box further comprises a medicine box C, and the medicine box C comprises another therapeutic agent.

27. A kit, comprising the pharmaceutical combination as defined in claim 1.
